(19) United States
(12) Patent Application Publication
(10) Pub. No.: US 2022/0243191 A1
(43) Pub. Date: Aug. 4, 2022

(54) METHOD FOR PRODUCING EXOSOMES BY ELECTRICAL STIMULATION

(71) Applicant: CELLAPEUTICS BIO, Gyeonggi-do (KR)

(72) Inventors: Kyeong-Kyu KIM, Seoul (KR); Gorai SUKHAMOY, Gyeonggi-do (KR)

(21) Appl. No.: 17/620,990

(22) PCT Filed: Jun. 22, 2020
(86) PCT No.: PCT/KR2020/008079

§ 371 Date: Dec. 20, 2021

(30) Foreign Application Priority Data
Jun. 20, 2019 (KR) 10-2019-0073405

(51) Int. Cl. C12N 13/00; C12N 5/079; C12M 1/42

(52) U.S. Cl. CPC C12N 13/00; C12M 35/02; C12N 5/0622

(57) ABSTRACT

The present disclosure relates to a method for producing exosomes through electrical stimulation. More particularly, the present disclosure relates to a method for producing exosomes, the method including: (a) applying radio frequency (RF) electrical stimulation to cells and culturing the cells; and (b) isolating exosomes from the cells and a culture medium containing the cells.

### TECHNICAL FIELD

The present disclosure relates to a method for producing exosomes through electrical stimulation. More particularly, the present disclosure relates to a method for producing exosomes, the method including: (a) applying radio frequency (RF) electrical stimulation to cells and culturing the cells; and (b) isolating exosomes from the cells and a culture medium containing the cells.

### BACKGROUND ART

Exosomes are small, membrane vesicles released from various types of cells. Exosomes are reported to range in diameter from around 30 to 300 nm. It was observed through electron microscopy that exosomes originate in specific compartments called multivesicular bodies (MVBs) in cells, rather than directly detach from the plasma membrane, and are released and secreted out of the cells. When the MVB fuses with the plasma membrane, small vesicles are released into the extracellular space, and it is called exosomes. Although the molecular mechanism for the formation of the exosomes is not known for certain, not only red blood cells but also various immune cells including macrophages, B-lymphocytes, T-lymphocytes, dendritic cells, platelets, tumor cells, and stem cells are known to produce and secrete exosomes while they are alive.

It is known that physical signals such as heat, sound waves, lasers, oxidative stress, and LED light can alter relative gene and protein expression in biological cells. Therefore, the physical stimuli listed for example above may change the secretion or functionality of exosomes. For example, it has been reported that upregulation of heatshock protein Hsp/c70 by heat shock can enhance exosome secretion from astrocytes.

However, the technology for improving the secretion and functionality of exosomes by applying electrical stimulation to cells or tissues is not advanced enough, and a technology for increasing exosome production and secretion is required.

### DISCLOSURE

### Technical Problem

The inventors found and confirmed that the application of radio frequency electrical stimulation to cells enhances the production efficiency and functionality of exosomes.

An objective of the present disclosure is to provide a method for producing exosomes, the method including: (a) applying radio frequency electrical stimulation to cells and culturing the cells; and (b) isolating exosomes from the cells and a culture medium containing the cells.

Another objective of the present disclosure is to provide an exosome culture apparatus including: a radio frequency (RF) generator for applying radio frequency of 3 KHz to 300 GHz; a culture chamber; and electrodes attached to respective ends of the culture chamber.

The effects, features, and objectives of the present disclosure are not limited to the ones mentioned above, and other effects, features, and objectives not mentioned above can be clearly understood by those skilled in the art from the following description.

### Technical Solution

In order to achieve the objective, the present disclosure provides a method for producing exosomes, the method including: (a) applying radio frequency electrical stimulation to cells and culturing the cells; and (b) isolating exosomes from the cells and a culture medium containing the cells.

In addition, the present disclosure provides a method of enhancing the production and secretion of exosomes by applying radio frequency (RF) electrical stimulation to cells.

In the present disclosure, the cells may be animal cells or plant cells. When the cells are animal cells, the cells may refer to cells derived (isolated) from mammals. Herein, the mammals may include: primates such as humans or monkeys, and rodents such as rats and mice. Preferably, the mammal may refer to a rabbit, dog, monkey, horse, cat, goat, mouse, rat, pig or human, and the mammalian cell may be a somatic cell, a germ cell, or a cancer cell. The plant may mean corn, rice, cotton, wheat, or the like, but is not limited thereto.

In another embodiment, the cells may include one or more cells selected from the group consisting of: human tissue-derived somatic cells including neurospheres, fibroblasts, epithelial cells, muscle cells, cardiac cells, kidney cells, nerve cells, hair cells, root hair cells, hair follicle cells, oral epithelial cells, beta cells, gastric mucosal cells, goblet cells, G cells, immune cells, and epidermal cells; cells extracted from solutions excreted from a human body, including urine, saliva, sweat, and blood; bone marrow-derived stem cells including nerve cord blood; adipose-derived stem cells; adult stem cells; and pluripotent stem cells including iPSC and embryonic stem cells.

In a further embodiment, the neurosphere may be one or more selected from the group consisting of Schwann cells, neurons, glia cells, astrocytes, and oligodendrocytes but may not be limited thereto.

In a yet further embodiment, the radio frequency electrical stimulation may be performed with radio waves having a frequency in a range of 0.05 to 3 MHz, 50 to 1000 KHz, 100 to 1000 KHz, 200 to 800 KHz, 200 to 600 KHz, 200 to 500 KHz, 250 to 500 KHz, 300 to 400 KHz, or 330 to 370 KHz, but the radio frequency range for the electrical stimulation may not be limited thereto. In one example of the present disclosure, the electrical stimulation is performed with radio waves with a frequency of 350 KHz.

In a yet further embodiment, as the exosome isolation technique, one or more methods selected from the group consisting of density gradient isolation, ultracentrifugation, filtration, dialysis, free flow electrophoresis, precipitation by polymers including PEG, trapping on an ELISA plate, an antibody-coated bead and Exoquick method.

In a yet further embodiment, the isolated exosome may be an exosome that is enhanced in activity by radio frequency electrical stimulation but may not be limited thereto.

Further, the present disclosure provides an exosome culture apparatus comprising: a radio frequency (RF) generator for applying radio waves having a frequency in a range of 3 KHz to 300 GHz; a culture chamber; and electrodes attached to respective ends of the culture chamber.

In one embodiment of the present disclosure, the culture apparatus may be an apparatus to improve the production and secretion of exosomes in cells but is not limited thereto.

In another embodiment of the present disclosure, the exosome culture apparatus may further include at least one selected from the group consisting of an oscillograph, a temperature sensor, a pH sensor, a DO sensor, a CO₂ sensor, an O₂ sensor, and a humidity sensor. However, the present disclosure is not limited thereto.

In a further embodiment of the present disclosure, the radio frequency (RF) generator may be a capacitive-resistance electric transfer (CRET) system, but is not limited thereto.

In a yet further embodiment of the present disclosure, the RF generator may be a device configured to generate radio waves having a frequency in a range of 0.05 to 3 MHz.

### Advantageous Effects

The inventors of the present application have developed a technology for producing exosomes in large quantities by promoting the production and secretion of exosomes without causing damage to cells and have found that the exosomes produced by the technology are higher in cell activity than those produced naturally in cells and thus can be used as a therapeutic agent for various diseases.

### DESCRIPTION OF DRAWINGS

FIG. 1 illustrates an exosome culture apparatus composed of a capacitive-resistance electric transfer (CRET) system for generating radio frequency waves, an oscillograph, a temperature sensor, electrodes, and a culture chamber that is 8 cm in length and 2 cm in diameter;

FIG. 2A illustrates the yield of exosomes when purified by ultracentrifugation (UC), a PEG method (PEG), and a commercially available kit;

FIG. 2B illustrates the yield of exosomes in a RF treated group (RF) and an RF untreated group (W/O RF);

FIG. 3A is a Transmission Electron Microscope (TEM) image showing the shape of the exosomes;

FIG. 3B is a dynamic light scattering (DLS) result showing the size distribution of exosomes;

FIG. 3C shows the results of a western blot to check whether exosomes express CD63 and CD81;

FIG. 4 is a picture observed under a fluorescence microscope, whether exosomes generated from Schwann cells are internalized into the NSC34 motor neuron cell line;

FIG. 5A shows the picture of observation of the neurite growth of the NSC34 cell line in conditioned media in which Schwann cell-derived exosomes isolated by different methods are contained, and in a control medium, in which the degree of neurite growth for each medium was evaluated on the basis of the length of the neurite measured under a microscope;

FIG. 5B shows the result of comparison in the neurite growth of the NSC34 cell line among the conditioned media in which Schwann cell-derived exosomes isolated by different methods are contained and the control medium;

FIG. 5C illustrates a result showing the change in the length of neurites when treated with exosomes isolated by different methods, in which the X-axis represents length distribution (length in micrometer) and the Y-axis represents the number of cells having the neurite length represented by the X-axis;

FIG. 6A is a diagram illustrating the quantification test results of Schwann cell-derived exosomes produced under an RF treated condition (RF) and an RF untreated condition (W/O RF);

FIG. 6B illustrates the result of observation of the neurite growth of the NSC34 cell treated with Schwann cell-derived exosomes produced under an RF treated condition (RF) and an RF untreated condition (W/O RF), in which the X-axis represents length distribution (length in micrometer) and the Y-axis represents the number of cells having the neurite length represented by the X-axis;

FIG. 7 is a diagram illustrating the quantification test results of HEK293 cell-derived exosomes produced under an RF treated condition (RF) and an RF untreated condition (W/O RF); and

FIG. 8 is a diagram showing the quantification test results of L929 cell-derived exosomes produced in an RF treated condition (RF) and an RF untreated condition (W/O RF).

### BEST MODE

The present disclosure provides a method for producing exosomes, the method including: (a) applying radio frequency electrical stimulation to cells and culturing the cells; and (b) isolating exosomes from the cells and a culture medium containing the cells.

### MODE FOR INVENTION

The present disclosure provides a method for producing exosomes, the method including: (a) applying radio frequency electrical stimulation to cells and culturing the cells; and (b) isolating exosomes from the cells and a culture medium containing the cells.

In the present disclosure, the cells may be animal cells or plant cells. When the cells are animal cells, the cells may refer to cells derived (isolated) from mammals. Herein, the mammals may include: primates such as humans or monkeys; and rodents such as rats and mice. Preferably, the mammal may refer to a rabbit, dog, monkey, horse, cat, goat, mouse, rat, pig or human, and the mammalian cell may be a somatic cell, a germ cell, or a cancer cell. The plant may mean corn, rice, cotton, wheat, or the like, but is not limited thereto.

In the present disclosure, the cells may include one or more cells selected from the group consisting of: human tissue-derived somatic cells including neurosphere, fibroblast, epithelial cells, muscle cells, cardiac cells, kidney cells, nerve cells, hair cells, root hair cells, hair follicle cells, oral epithelial cells, beta cells, gastric mucosal cells, goblet cells, G cells, immune cells, and epidermal cells; cells extracted from solutions excreted from a human body, including urine, saliva, sweat, and blood; bone marrow-derived stem cells including nerve cord blood; adipose-derived stem cells; adult stem cells; pluripotent stem cells including iPSC and embryonic stem cells.

In the present disclosure, the neurosphere may be one or more selected from the group consisting of Schwann cells, neurons, glia cells, astrocytes, and oligodendrocytes but may not be limited thereto.

Hereinafter, the method according to the present disclosure will be described step by step in detail.

<(a) Applying radio frequency electrical stimulation to cells and culturing cells>

In one example of the present disclosure, exosomes were secreted by applying radio frequency electrical stimulation to Schwann cells and HEK293 cells, and it was confirmed that the number of the secreted exosomes was significantly increased compared to a control group without applying radio frequency electrical stimulation.

In addition, it was confirmed that exosomes secreted by applying radio frequency electrical stimulation to Schwann cells promote neurite growth of motor neurons compared to exosomes secreted under the condition without an RF electrical stimulation.

Conventional stimulation was a single kind of stimulation such as a physical stimulation, whereas stimulation used in the present disclosure includes multiple kinds of stimuli using radio frequency electrical stimulation which is a combination of an electromagnetic energy stimulation and a physical stimulation, thereby effectively inducing physiological or physical changes in cells, resulting in that the production and secretion of exosomes are boosted.

The boost in the production and secretion of exosomes by the method of the present disclosure is caused by a physical wave stimulation of radio frequency during applying of the radio waves, a stimulation by energy generated due to electromagnetic conversion in an electric field generated during the application of the radio waves to cells, and gene structural and physiological changes in the cells, which are caused due to the resulting change in polarity inside the electric field.

When the radio frequency electric stimulation is applied to a cell, an electric field is formed around the cell, and electromagnetic energy may be generated in the electric field. Although the electromagnetic energy does not cause direct flow of electron charges, it may cause an energy flow similar to movement of electrons, thereby inducing transfer of various substances according to the polarity of each substance in the cell, and electric waves generated by the radio frequency also may induce changes of intracellular substances. The electromagnetic energy and the physical energy induce changes in gene structural and physiological substances in cells to change the biochemical and physiological properties of the cells, thereby promoting the production of exosomes and the release of the generated exosomes.

In the present disclosure, the Schwann cells refer to glial cells of the peripheral nervous system. The Schwann cells not only play an important role in the generation and differentiation of nerves but also plays a key role in axon regeneration and remyelination when nerves are damaged. Specifically, a bundle of axons is called a nerve fiber. Schwann cells form a nerve fiber sheath which is the outermost membrane surrounding the axon. Nerve fibers myelinated by the Schwann cells exhibit a fast nerve conduction speed because the myelin acts as an insulator. On the other hand, when there is damage to the myelin, the conduction speed is slowed and secondary damage to the axon occurs due to demyelination.

As a culture medium used in the present disclosure, any existing basal medium known in the related art may be used without limitation. As the basal medium, an artificial medium may be synthesized or a commercially prepared medium may be used. Examples of commercially prepared media include Dulbecco's Modified Eagle's Medium (DMEM), Minimal Essential Medium (MEM), Basal Medium Eagle (BME), RPMI 1640, F-10, F-12, α-Minimal essential Medium (α-MEM), Glasgow's Minimal Essential Medium (G-MEM), and Iscove's Modified Dulbecco's Medium. However, the culture medium that can be used may not be limited thereto. Among them, a DMEM medium may be used.

Examples of the culture media for cell culture used in the present disclosure include all of the culture media commonly used for cell culture in the related art. The culture medium used for cell culture generally contains a carbon source, a nitrogen source, and a trace component.

The term “culture” used therein refers to all actions performed to grow cells or microorganisms in appropriately artificially controlled environmental conditions. The optimal temperature for the culture may range from 30° C. to 50° C., preferably from 30° C. to 45° C., and most preferably 35° C. to 40° C., but the temperature for the culture is not limited thereto. The duration for the culture may range from 36 to 60 hours, preferably from 40 to 56 hours, more preferably from 44 to 52 hours, and most preferably from 46 to 50 hours, but is not limited thereto. In one example of the present disclosure, media were collected after 48 hours of culturing after application of radio frequency electrical stimulation.

In the present specification, the term “exosomes” are membrane-structured vesicles secreted from various types of cells, and are known to play various roles. For example, an exosome fuses with another cell or tissue to deliver membrane components, proteins, and RNAs.

In the present specification, the exosomes may be exosomes produced by application of radio frequency electrical stimulation to cells and enhanced in functionality or activity thereof, but are not limited thereto. In an example of the present disclosure, it was confirmed that the neurites growth ability of the Schwann cell-derived exosomes produced with application of radio frequency electrical stimulation to the cells was superior to that of the exosomes produced without application of radio frequency electric stimulation to the cells. It was confirmed that the activity of exosomes derived from specific cells was enhanced. That is, the isolated exosomes may have enhanced exosome activity but are not limited thereto.

In the present specification, the term “radio frequency electrical stimulation” is collectively referred to as radio frequency (RF) and means an electromagnetic wave having a frequency of 3 KHz to 300 GHz, or a wavelength of 100 km to 1 mm. Radio frequency electrical stimulation corresponding to radio frequency (RF) has been utilized for transmitting and receiving radio waves used for broadcasting and various wireless communication signals, but the use of radio frequency for generating function-enhanced exosomes in large quantities was first developed by the inventors of the present application. Radio frequency for electrical stimulation can be classified as follows according to frequency.

TABLE 1

| Frequency | Wavelength | Name | Abbreviation |
|---|---|---|---|
| 3~30 KHz | 100~10 km | Very low frequency | VLF |
| 30~300 KHz | 10~1 km | Low frequency | LF |
| 300 KHz~3 MHz | 1 km~100 m | Medium frequency | MF |
| 3~30 MHz | 100~10 m | High frequency | HF |
| 30~300 MHz | 10~1 m | Very high frequency | VHF |
| 300 MHz~3 GHz | 1 m~10 cm | Ultra high frequency | UHF |
| 3~30 GHz | 10~1 cm | Super high frequency | SHF |
| 30~300 GHz | 1 cm~1 mm | Extremely high | EHF |
| | | frequency | |

The radio frequency alternating electrical stimulation used the present disclosure may be a low frequency or a medium frequency. Preferably, it may be a medium frequency.

Preferably, it may have a frequency in a range of from 0.05 to 3 MHz, from 50 KHz to 1000 KHz, from 100 to 1000 KHz, from 200 to 800 KHz, from 200 to 600 KHz, from 200 to 500 KHz, from 250 to 500 KHz, from 300 to 400 KHz, or from 330 to 370 KHz, but the frequency range of the radio frequency for the electrical stimulation may not be limited thereto. In one example of the present disclosure, the electrical stimulation is performed with a frequency of 350 KHz.

<(b) Isolation of Exosomes from Cells and Culture Medium Containing Cells>

The step of isolating exosomes may be performed by one or more methods selected from the group consisting of density gradient isolation, ultracentrifugation, filtration, dialysis, free flow electrophoresis, polymer based precipitation including PEG, trapping on an ELISA plate, an antibody-coated bead and Exoquick method.

Several techniques for isolation or purification of extracellular exosomes have been described above. These methods may include fractional centrifugation which includes an ultracentrifugation step (Thery et al. 2006); affinity chromatography (Taylor & Gercel-Taylor, 2008); and polymer-mediated precipitation (Taylor et al. 2011). Here, the ultracentrifugation step uses polyethylene glycols (PEG) having different molecular weights, including: a whole exosome isolation reagent from Life Technologies Corporation (U.S. Pat. No. 8,901,284) and ExoQuick™ (U.S. Pat. Application Publication No. 2013/0337440 A1); and typical entrapment on defined pore size membranes (Grant et al. 2011) such as ExoMir™ (U.S. Pat. No. 2013/0052647 A1) using two filters having different pore sizes and connected in series.

In one example of the present disclosure, the inventors of the present application performed tests to find the optimal separation/purification method by using the ultracentrifugation, the PEG method, and the ExoQuick method, and has found that the PEG method is most suitable in terms of cost and efficiency.

The present disclosure also provides an exosome culture apparatus including: a radio frequency (RF) generator for applying radio frequency waves having a frequency in a range of 3 KHz to 300 GHz; a culture chamber; and electrodes attached to respective ends of the culture chamber.

The apparatus may be characterized in that it enhances the production and secretion of exosomes in cells, but the function of the apparatus is not limited thereto.

The exosome culture apparatus may further include one or more devices selected from the group consisting of an oscillograph, a temperature sensor, a pH sensor, a DO sensor, a CO₂ sensor, an O₂ sensor, and a humidity sensor, but the elements of the exosome culture apparatus are not limited thereto.

In the present specification, the term “oscillograph” refers to a device for observing and recording temporal changes in current, voltage, and frequency. The oscillographs are divided into electromagnetic types, cathode linear types, etc. A figure recorded by an oscillograph is called an oscillogram. In one example of the present disclosure, an oscillograph is used to record the frequency and output (power).

In the present specification, the term “radio frequency (RF) generator” refers to a device for generating an alternating electrical stimulation of a radio wave frequency. The RF generator used in the present disclosure uses a capacitive resistance electric transfer (CRET) system, but the present disclosure is not limited thereto. That is, the RF generator used in the present disclosure may be a CRET system, but the present disclosure is not limited thereto.

In the present specification, the range of the radio frequency (RF) for electrical stimulation may be from 0.05 to 3 MHz, 50 to 1000 KHz, 100 to 1000 KHz, 200 to 800 KHz, 200 to 600 KHz, 200 to 500 KHz, 250 to 500 KHz, 300 to 400 KHz, or 330 to 370 KHz, but the radio frequency range for the electrical stimulation may not be limited thereto. In one example of the present disclosure, a radio frequency having a frequency of 350 KHz is applied for electrical stimulation.

In one example of the present disclosure, an RF generator that generates waves in a frequency range of 0.05 to 3 MHz and an output power range of 10 to 50 W was used.

In the present specification, the term “temperature sensor” refers to a device for observing and/or sensing the temperature of the culture chamber.

In the present specification, the term “culture chamber” refers to a container for providing a space for culturing cells. In the culture chamber, the cells are cultured with radio frequency waves applied thereto for electrical stimulation.

In the present disclosure, the cells cultured in the culture chamber may include one or more cells selected from the group consisting of: human tissue-derived somatic cells including neurosphere, fibroblast, epithelial cells, muscle cells, cardiac cells, kidney cells, nerve cells, hair cells, root hair cells, hair follicle cells, oral epithelial cells, beta cells, gastric mucosal cells, goblet cells, G cells, immune cells, and epidermal cells; cells extracted from solutions excreted from a human body, including urine, saliva, sweat, and blood; bone marrow-derived stem cells including nerve cord blood; adipose-derived stem cells; adult stem cells; pluripotent stem cells including iPSC and embryonic stem cells.

The culture chamber may be designed to have a predetermined working volume. The culture chamber is not limited in its shape, but may preferably have a cylindrical shape.

In the present specification, the term “electrodes attached to respective ends of the culture chamber” may refer to electrodes for applying radio frequency waves for electrical stimulation in the culture chamber. The electrodes may be made of one or more materials selected from the group consisting of platinum, gold, copper, palladium and titanium, but the material of the electrodes is not limited thereto.

In addition, the electrodes attached to respective ends of the culture chamber may be coated with polyamide for the purpose of heat transfer, but the configuration of the electrodes is not limited thereto. In one example of the present disclosure, cells were cultured with the exosome culture apparatus disclosed above in which radio frequency waves are applied to the cells for electrical stimulation. As a result, it was confirmed that the yield of production of exosomes was significantly increased and the activity of the produced exosomes was superior to that of naturally occurring exosomes.

Hereinbelow, preferred examples will be described to aid in understanding the present disclosure.

However the examples described below are provided only to facilitate the understanding of the present invention and thus the details in the examples should not be construed to limit the scope of the present invention.

### Example 1 Experimental Preparation and Experimental Method

### 1-1. Radio Frequency Wave Electrical Stimulation Treatment for Schwann Cells

Schwann cells were exposed to a radiofrequency (RF) electrical stimulation generated by a capacitive resistance electric transfer (CRET) system. As the CRET system, an E-motion Plus TM303 system (manufactured by Plus, Seoul, Korea) was used. This system generates an RF alternating electrical stimulation in a frequency range of 0.05 to 0.50 MHz and an output power range of 10 to 50 W. This RF generator was fundamentally similar to Indiva Active HCR 902 (INDIBA, Barcelona, Spain) which was commonly used for medical treatment, and the frequency and output power of the RF generator were measured with a Tektronix oscilloscope TDS 210 (Beaverton, Oreg., USA).

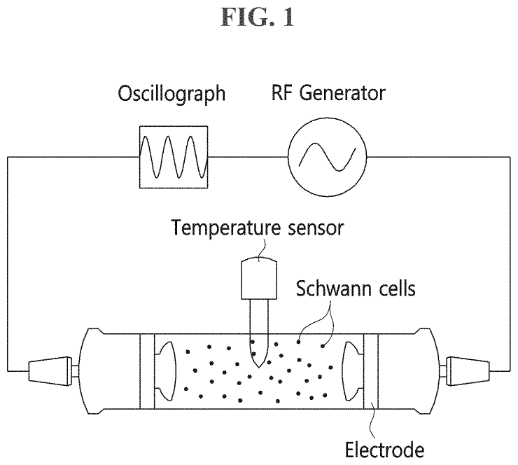

Referring to FIG. 1, electrodes attached to respective ends of a culture chamber including a glass tube that was 8 cm in length and 2 cm in diameter were coated with polyamide for the purpose of heat transfer, and the electrodes were connected to the RF generator.

5×10⁶ human Schwann cells (hSc) were placed in 10 ml a Schwann cell culture medium. The cells were exposed to a continuous RF flow for 15 minutes at a pulse rate of 30 seconds at a constant temperature of 37° C. The glass tube was connected by two electrodes at the respective ends of the tube, capacitive coupling. Equal numbers of RF-treated and RF-untreated cells were separately cultured in exosome-depleted Schwann cell media. After 48 hours of culturing, the conditioned media were collected and the produced exosomes were concentrated through a polyethylene glycol (PEG) method.

### 1-2. Exosome Purification and Characterization

Exosomes were isolated from the collected conditioned media and were purified using three different purification methods (ultracentrifugation, previously reported polyethylene glycol-6000, and the ExoQuick-TC PLUSTM kit). The initial stages were the same for all the purification methods.

After 48 hours of culturing, the conditioned medium was sequentially centrifuged at 300 g for 10 minutes, at 1,000 g for 10 minutes, and at 10,000 g at 4° C. for 30 minutes to remove live cells, dead cells, and debris. After the centrifugation, the supernatant was collected and the exosomes were concentrated by high-speed ultracentrifugation (with a type 70 Ti rotor manufactured by Beckman Coulter Inc.) at 100,000 g at 4° C. for 90 minutes. Pellets were collected and washed with PBS and the same centrifugation step was repeated. Finally, for long-term use, the pellets were resuspended in 100 μl of ice-cold PBS and stored at −80° C.

When using a polyethylene glycol (PEG) method, a 10% PEG-6000 solution (final concentration) was directly added and mixed with the supernatant collected in the centrifugation step. The mixed solution was incubated overnight at 4° C. After 12 hours of incubation, centrifugation was performed at the maximum speed at 4° C. for 1 hour in a tabletop centrifuge (manufactured by Eppendorf, model 5810R with S-4-104 swing bucket rotor; 3,214 g). Next, the supernatant was then decanted, dried for 5 minutes, and tapped occasionally to completely remove residual PEG. The resulting pellets were resuspended in 5 ml PBS. To obtain purer exosomes, this step was repeated once more. Finally, the pellets were suspended in 500 μl of particle-free PBS (pH 7.4).

An Exo-Quick-TC PLUSTM method (Kit method) is very simple. Exosomes were concentrated according to the protocol provided with the kit. The conditioned media was collected and the same initial centrifugation step described above was performed to remove cell debris and dead cells. Next, an exo-precipitation solution was added to the conditioned media and the cells were incubated at 4° C. overnight. Next, the solution was centrifuged and exosomes were down-pelletized. The pellets were maintained in a resuspension buffer and added to beads for further purification. Finally, centrifugation was performed at 8,000 g for 5 minutes and then the supernatant was collected.

### 1-3. Western-Blot Assay

Exosome marker proteins were detected by western blot analysis according to the standard protocols (Lopez-Verrilli, Picou, & Court, 2013). Purified exosomes were disrupted using a radio immunoprecipitation assay (RIPA) buffer supplemented with 1 mM PMSF, which is a protease inhibitor. The solution was incubated at room temperature for 30 minutes. For the western blot analysis, exosome samples were separated with 10% SDS-PAGE and transferred to a polyvinylidene fluoride (PVDF) membrane. The membrane was blocked in Tris-buffered saline (TBST) containing 5% non-fat skim milk at room temperature for 1 hour and was then washed. After three-step washing with TBST, the membrane was incubated with antibodies containing CD63, CD81, and TSG101 at 4° C. overnight, followed by three-step washing with TBST. The membrane was then incubated with an appropriate anti-HRP conjugate and the bands were visualized with a chemiluminescent reagent and detected using a ChemiDoc system.

### 1-4. Dynamic Light Scattering (DLS) Analysis

The size distribution of the purified exosomes was measured through dynamic light scattering (DynaPro, NanoStar). 10 μl of a diluted exosome solution was placed in a cuvette, and a size distribution experiment was performed for each exosome preparation.

### 1-5. Transmission Electron Microscopy (TEM)

The shape of the exosomes was analyzed through TEM. Sample preparation was performed according to standard protocols. 4% paraformaldehyde was added to 10 μl of the exosome solution. 5 μl of the solution was placed on a grid coated with Formvar-carbon and incubated for 20 minutes in a dry environment. To wash off the excess solution, the grid was transferred with clean forceps to a PBS drop. The grid was then transferred to 50 μl of glutaraldehyde and washed with water several times (for example, 7 to 10 times). For contrast of samples, 50 μl of a uranyl-oxalate solution was placed on a grid, incubated for 5 minutes, and then inserted into a mixture of 4% uranyl acetate and 2% methyl cellulose (mixing ratio=1:9). Finally, the grid was incubated with a methylcellulose solution on ice for 10 minutes and stored in a dry place so that the grid can be dried completely.

### 1-6. Cellular Uptake Assay

To study cellular uptake, purified exosomes were labeled with an ExoGlow™ Membrane EV labeling kit according to the manufacturer's protocol. 2 μl of a red dye was mixed with 12 μl of a reaction buffer. The dye was uniformly mixed by vortexing, and the mixture was added directly to 50 μg of the exosome solution and incubated for 30 minutes. Free dye was removed by desalting a spin column at 10,000 rpm for 1 minute. 10 μl of the labeled exosomes were collected and directly added to NSC34 cells to monitor internalization. After 6 hours, the used medium was removed and a fresh medium was added. Internalized exosomes were visualized by fluorescence microscopy.

### 1-7. Quantification of Exosomes

Purified exosomes were quantified using a commercially available Exocet Quantification Assay kit (Exocet 96A-1, System Biosciences). 20-30 μg of total protein containing exosomes were lysed with a lysis buffer included in the kit. The mixture solution was warmed at 37° C. for 5 minutes to liberate the exosome proteins. After a brief time of vortexing, the mixture solution was centrifuged at 1500×g for 5 minutes. The resulting supernatant was decanted into a separate tube. Then, 50 μl of the sample was mixed with 50 μl of a reaction buffer (A+B) and incubated in a 96-well plate at room temperature for 20 minutes. After the incubation, absorbance was measured with a spectrophotometer at 405 nm. In parallel experiments, standard curves were prepared using the standard solutions included in the kit.

### 1-8. Activity Determination

5×10⁴ cells were cultured in 1% FBS containing a DMEM medium on 6-well plates coated with poly-L-lysine (PLL). The following day, the cells were treated with exosomes containing the same amount of an hSc-conditioned medium as the hSc-exo. When treating exosomes, ˜9×10⁸ exosomes were used. After 48 hours of the treatment, neurite growth was observed.

### Example 2 Evaluation of Exosome Purification Methods

Exosome purification methods were optimized in terms of quality, size distribution, and production yield of exosomes.

In three different purification methods, ultracentrifugation (UC), exosome purification polyethylene glycol (PEG-6,000; PEG) and a commercially available kit were used, respectively.

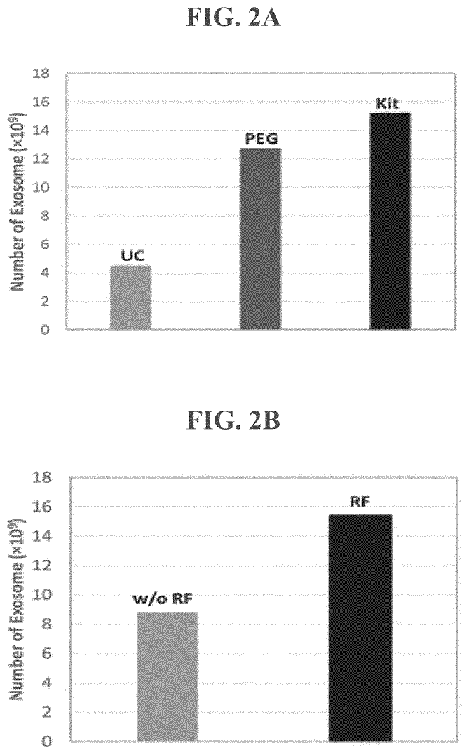

Referring to FIG. 2A, the exosome quantification showed that the commercially available kit purification method and the PEG purification method exhibited 4 times and 3 times higher exosome production yields than the UC purification method.

An RF frequency of 350 KHz was applied, the purification was performed through the PEG method, and the yield of production exosomes was checked.

Referring to FIG. FIG. 2B, an RF-treated group showed a production yield about 1.7 times greater than that of an RF-untreated group.

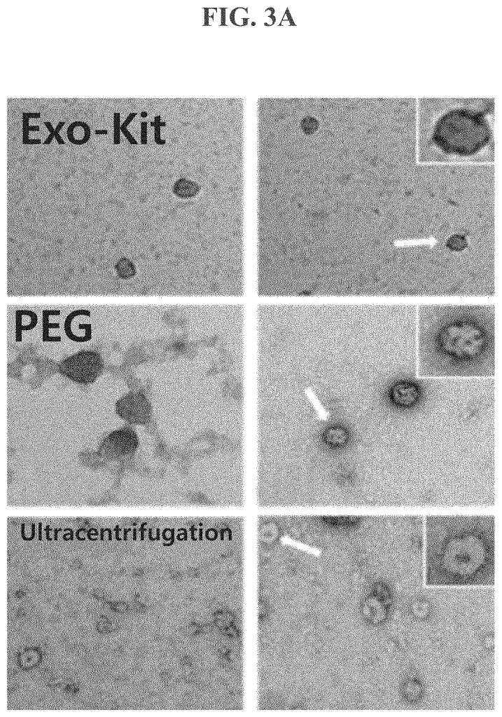

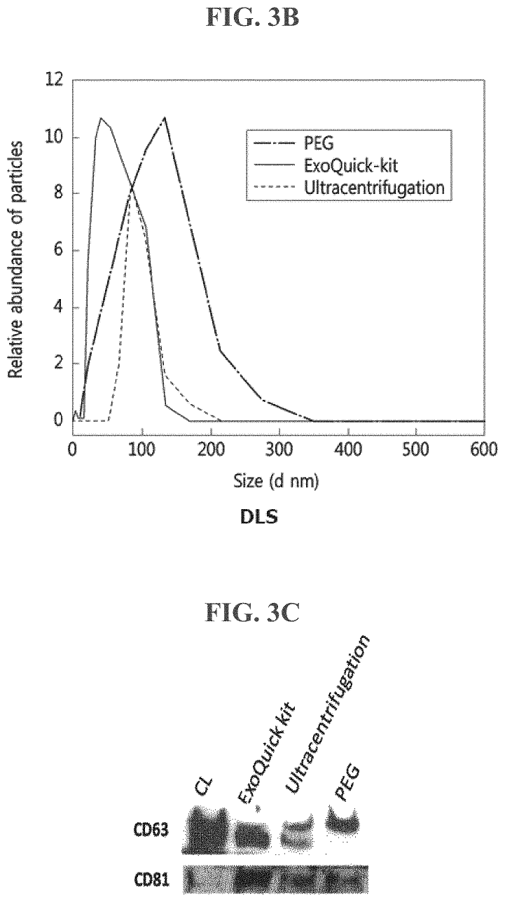

Referring to FIG. 3A, TEM images were used to observe the shapes of exosomes.

Referring to FIG. 3B, the size distribution of each exosome preparation was determined using dynamic light scattering (DLS) analysis. The sizes of the exosomes purified by the Kit purification method were uniquely homogenized, and the exosomes purified by the other two methods showed fairly non-uniform distributions within a reported size range (30 to 200 nm).

Referring to FIG. 3C, the western blot analysis showed that exosomes were present, and the expression of CD63 and CD81 showed that the Schwann cell-derived exosomes were successfully purified.

### Example 3 Schwann Cell Exosome Activity Assessment

To determine whether cellular factors for neurite growth can be delivered by exosomes, the inventors first investigated internalization of exosomes into NSC34 cells.

Purified exosomes were labeled with a commercially available EV membrane staining kit, ExoGlow™, and then incubated along with NSC34 at 37° C. for 6 hours.

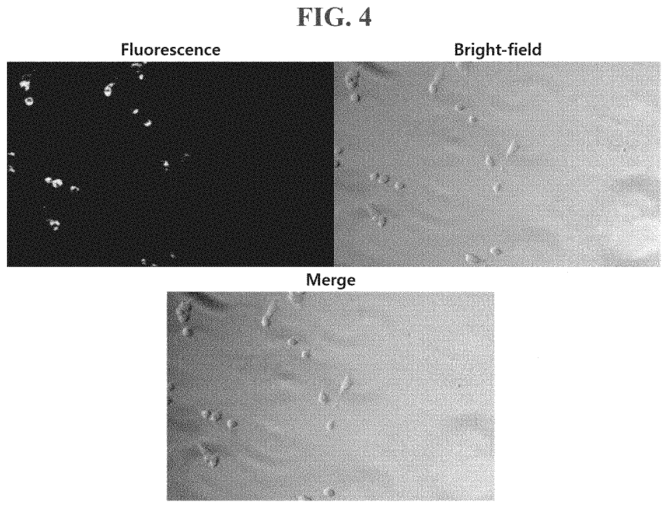

As shown in FIG. 4, the microscopic image showed that the exosomes were internalized into cells.

Next, the effect of each exosome preparation method on exosomes was investigated. The same number of exosomes (equal to or less than 9×10⁸) and each conditioned medium were added to NSC34 cells and cultured for 48 hours to induce neurite growth.

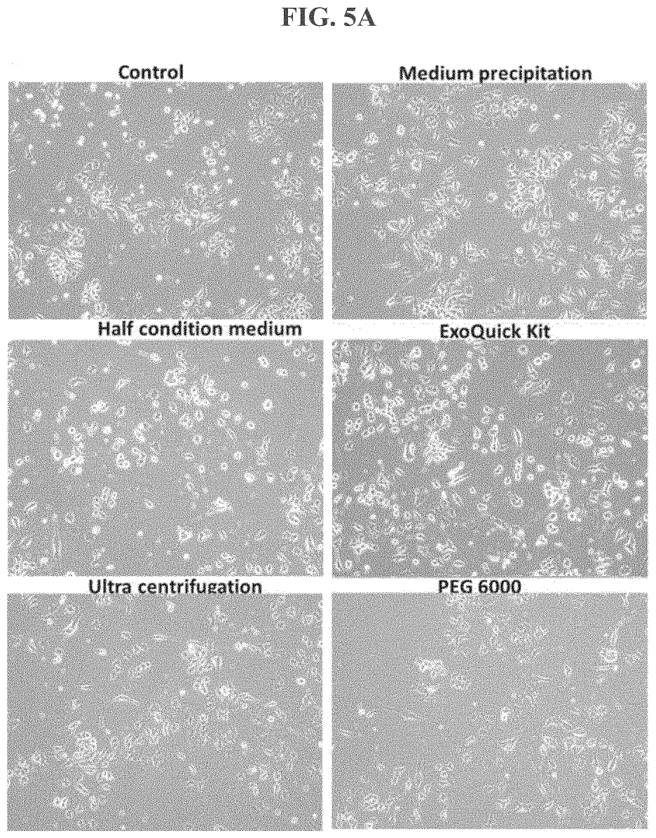

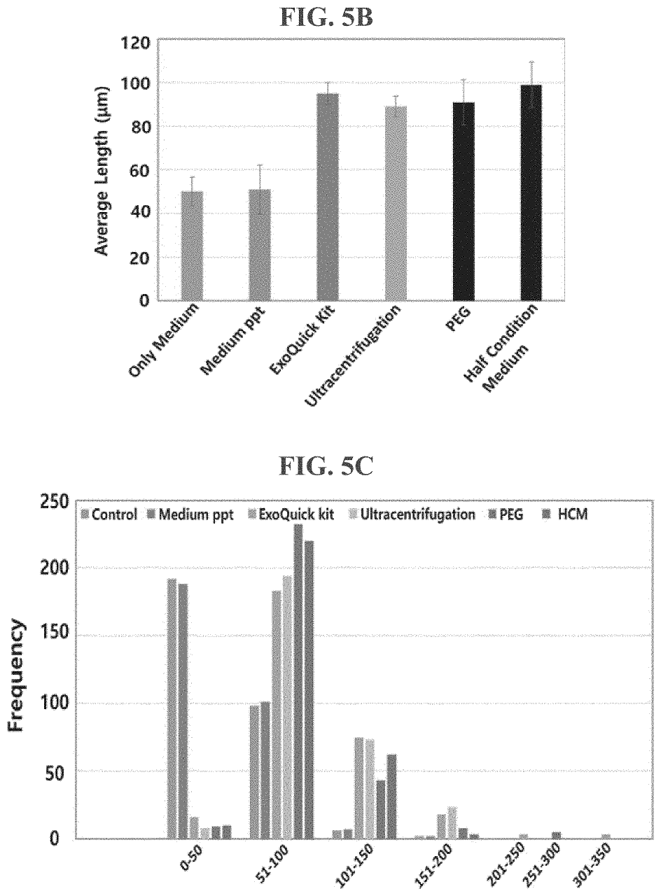

As shown in FIG. 5A, when comparing the neurite growth activity of exosomes purified by various exosome isolation methods, it was found that exosomes purified using the kit-based method showed the highest neurite growth activity.

As shown in FIG. 5B, the exosomes prepared by the UC-based, PEG-based, and kit-based methods showed 1.81, 1.83, and 1.93 fold neurite growth, respectively, compared to the control. This means that the exosomes prepared by the UC-based and PEG-based methods exhibited similar activity to the exosomes prepared by the kit-based method.

When all the results were combined, it was concluded that the PEG-based method was as efficient as the UC-based method and the commercially available kit-based method in terms of activity and production yield of exosomes.

As shown in FIG. 5C, it was confirmed that exosomes produced by applying RF to cells form many cells with long axons regardless of purification methods compared to the RF untreated group.

Therefore, the PEG-based method was used for all exosome sample preparations in subsequent experiments because the PEG-based method showed a production yield similar to that of the kit-based method at minimal cost.

### Example 4 Induction of Exosome Secretion by Alternating RF electrical stimulation

### Example 4-1 Induction of Exosome Secretion in Schwann Cells

There are several studies showing that the expression levels of miRNA, RNA, protein, lipid, and other factors in biological cells vary depending on physical and chemical stresses, and these factors are directly secreted outside the cells or secreted via exosomes that play an important role in many aspects.

Human Schwann cells (hSc) are known to release exosomes that play an important role in the regeneration and growth of neurons.

In this regard, the inventors observed whether radiofrequency (RF), known as a physical stress inducer, could enhance the release of exosomes from human Schwann cells (hSc).

To induce the release of exosomes, RF stimulation (frequency 350±3 KHz, power 42W) was applied to hSc for 15 min at 37° C., the cells were then cultured in a hSc growth medium for 48 hours, and exosomes were then harvested using an ExoCet kit.

In parallel experiments, the same number of RF untreated cells were cultured in the same growth medium.

As a result, it was found that RF-treated cells secrete 1.5-fold and 1.35-fold more exosome-containing proteins and RNA than RF-untreated cells.

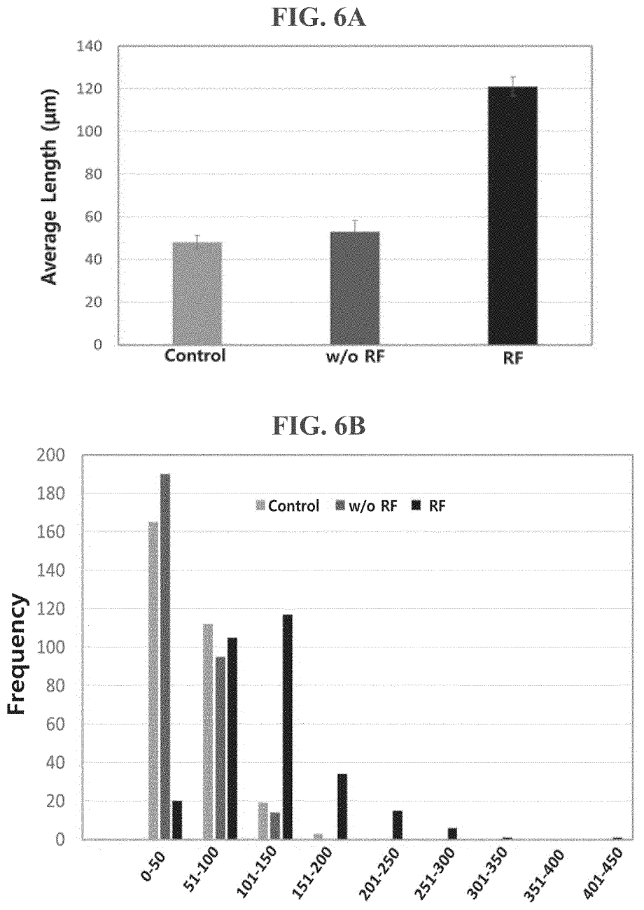

As shown in FIG. 6A, the exosome quantification experiment showed that the RF-treated cells released about 1.75 times more exosomes than that in the null experiment.

As shown in FIG. 6B, as a result of determining the neurite growth of the NSC 34 cells, the RF-treated exosomes (RF-exo) showed 1.25 times better neurite growth than RF-untreated exosomes (w/o RF-exo). Thus, it was confirmed that exosomes produced by applying RF form more cells with long axons. That is, it was confirmed that the activity of exosomes in the RF-treated group was superior to that in the RF-untreated group.

### Example 4-2 Induction of Exosome Secretion in HEK293 Cells

To induce the release of exosomes, RF stimulation (frequency 350±3 KHz) was applied to HEK293 cells for 15 min at 37° C., the cells were then cultured in a HEK293 cell growth medium for 48 hours, and exosomes were then quantified using an ExoCet kit.

In parallel experiments, the same number of RF-untreated cells were grown in the same growth medium.

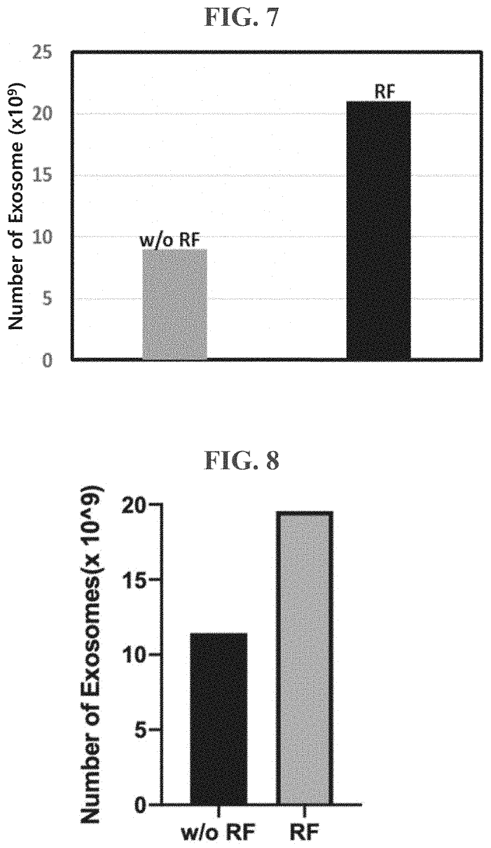

As a result, as shown in FIG. 7, it was found that the RF-treated cells secreted 2.3 times more exosomes than the RF-untreated cells.

### Example 4-3 Induction of Exosome Secretion in L929 Cells

To induce the release of exosomes, RF stimulation (frequency 350±3 KHz) was applied to L929 cells for 15 min at 37° C., the cells were then cultured in a L929 cell growth medium for 48 hours, and exosomes were then quantified using an ExoCet kit.

In parallel experiments, the same number of RF-untreated cells were grown in the same growth medium.

As a result, as shown in FIG. 8, it was found that the RF-treated cells secreted 1.7 times more exosomes than the RF-untreated cells.

Combining these results, it was confirmed that the RF electrical stimulation generated by the CRET system could enhance the exosome secretion from the cells, and the activity of the secreted exosome was also improved.

Therefore, the method of the present disclosure may be used for mass production of cell-derived exosomes that can be used for therapeutic purposes for various diseases.

While examples of the present disclosure have been described for the illustrative purposes, those skilled in the art will appreciate that the present disclosure can be implemented in other different forms without departing from the technical spirit or essential characteristics of the present disclosure. Therefore, it can be understood that the examples described above are only for illustrative purposes and are not restrictive in all aspects.

1. A method of producing exosomes, the method comprising: (a) applying radio frequency (RF) for electrical stimulation to cells and culturing the cells; and (b) isolating exosomes from the cells and a culture medium containing the cells.

2. The method according to claim 1, wherein the method enhances production and secretion of exosomes in the cells.

3. The method according to claim 1, wherein the cells are cells derived from mammals including human beings.

4. The method according to claim 1, wherein the cells include one or more cells selected from the group consisting of: human tissue-derived somatic cells including neurosphere, fibroblast, epithelial cells, muscle cells, cardiac cells, kidney cells, nerve cells, hair cells, root hair cells, hair follicle cells, oral epithelial cells, beta cells, gastric mucosal cells, goblet cells, G cells, immune cells, and epidermal cells; cells extracted from solutions excreted from a human body, including urine, saliva, sweat, and blood; bone marrow-derived stem cells including nerve cord blood; adipose-derived stem cells; adult stem cells; and pluripotent stem cells including iPSC and embryonic stem cells.

5. The method according to claim 4, wherein the neurosphere is selected from the group consisting of Schwann cells, neurons, glial cells, astrocytes, and oligodendrocytes.

6. The method according to claim 1, wherein the RF electrical stimulation is application of waves having a frequency of 0.05 to 5 MHz.

7. The method according to claim 1, wherein the step (b) of isolating the exosomes is performed by one or more techniques selected from the group consisting of density gradient isolation, ultracentrifugation, filtration, dialysis, free flow electrophoresis, precipitation by polymers including PEG, trapping on an ELISA plate, an antibody-coated bead and Exoquick method.

8. The method according to claim 1, wherein the isolated exosomes have enhanced activity.

9. An exosome culture apparatus comprising: a radio frequency (RF) generator for applying radio frequency waves of 3 KHz to 300 GHz; a culture chamber; and electrodes attached to respective ends of the culture chamber.

10. The exosome culture apparatus according to claim 9, wherein the apparatus enhances production and secretion of exosomes in cells.

11. The exosome culture apparatus according to claim 9, wherein the RF generator is a capacitive resistance electric transfer (CRET) system.

12. The exosome culture apparatus according to claim 9, further comprising at least one selected from the group consisting of an oscillograph, a temperature sensor, a pH sensor, a DO sensor, a CO₂ sensor, an O₂ sensor, and a humidity sensor.

13. The exosome culture apparatus according to claim 9, wherein the RF generator is a device configured to generate RF waves having a frequency of 0.05 to 3 MHz.
